(19) United States
(12) Patent Application Publication
(10) Pub. No.: US 2024/0366439 A1
(43) Pub. Date: Nov. 7, 2024

(54) ABSORBENT UNDERPAD WITH DRAWINGS OBTAINED BY VACUUM AND ITS MANUFACTURING PROCESS

(71) Applicant: GHOST - CORPORATE MANAGEMENT, S.A., Lisboa (PT)

(72) Inventor: José Nuno RODRIGUES RIBEIRO, Lisboa (PT)

(21) Appl. No.: 18/291,587

(22) PCT Filed: Jul. 26, 2021
(86) PCT No.: PCT/PT2021/050024

§ 371 Date: Jan. 24, 2024

(51) Int. Cl. A61F 13/84; A61F 13/15; A61F 13/47; B32B 3/30; B32B 5/02; B32B 7/12

(52) U.S. Cl. CPC A61F 13/84; A61F 13/15731; A61F 13/47; B32B 3/30; B32B 5/022; B32B 7/12; A61F 2013/8497; B32B 2255/02; B32B 2307/726

(57) ABSTRACT

The present invention refers to the production of an absorbent underpad with drawings or patterns on its absorbent surface, using a vacuum technique that distributes the absorbent raw material through the intended and predefined drawings or pattern on cut out molds (1), existing in the pockets (9) (10) of the forming wheel (2) of underpads,

This innovative technique differs from the current technical status, which transposes drawings to the absorbent underpads through embossing techniques, which consist of passing absorbent underpads through embossing rolls previously embossed with the drawings or patterns that are intended to be transposed onto the underpads.

### TECHNICAL FIELD OF THE INVENTION

The present invention is within the field of disposable absorbent articles, more specifically in the absorbent underpads and its manufacturing process.

### TECHNICAL STATUS

The absorbent underpads are disposable absorbent products with multiple uses.

The high supply of these products in the market leads to a constant need to innovate and differentiate products marketed, urging producers and marketers the increasingly need to differentiate their products, particularly through the customization of underpads by engraving differentiating designs or patterns on their products.

However, the prior art obtains this customization through a technique called embossing, implementing designs or patterns, pre-embossed on the surface of the embossing rollers, for the underpads, by passing these underpads between the embossing rollers, which compress the underpads and print these same designs or patterns on their embossed surface.

This technique, due to the compression it uses to obtain the designs or patterns, compresses the absorbent raw material, changing its characteristics and properties.

The present invention aims to solve this problem by creating an absorbent product with the inclusion of a defined design or pattern and without changing the appearance and characteristics of the final product.

### INVENTION SUMMARY

The disposable absorbent articles, such as underpads, are designed to absorb and retain or contain liquid and solid residues in order to prevent filth from the user's body and/or to protect the surface on which this underpad is applied.

The existing underpads, more specifically their absorbent surface, can be smooth or personalized with images or patterns engraved on them. This engraving of images or patterns is achieved by compression or compaction techniques, using compactor rollers or embossing rollers, using a technique called embossing.

However, this same technique commonly used by the industry affects the characteristics of the final product, because when engraving the intended design using compaction rollers or embossers, it simultaneously causes a compaction of the absorbent material itself, forming grooves throughout the underpad, making it more rigid and less pleasant to the touch or even contact with the human body.

Furthermore, the use of drawing or pattern engraving techniques on the absorbent underpad, through the use of embossing rolls, in addition to embossing on the absorbent part of the underpad, it also causes crushing of the absorbent raw material applied to the nonwoven topsheet and the backsheet, transposing this same recording to the backsheet, which is the impermeable part of the absorbent underpad.

This circumstance, in addition to having a visually negative impact, impairs the waterproofing characteristics of the backsheet, making it more fragile and making it more susceptible to possible breakage and leakage of residues that the underpad must protect against.

The innovative technique that is intended to patent allows to obtain a custom underpad with the design or pattern wished upon it, without affecting the characteristics of the underpad, including its absorption and its softness to the touch and the integrity of the backsheet.

To obtain product the aforementioned this with characteristics, molds were created, with designs or patterns that are intended to be transposed to the underpads, pre-cut in the referred molds. This technique is characterized by the transposition of the design or pattern existing in the mold of the forming wheel, previously selected, which, through a vacuum system present in this forming wheel, distributes the absorbent raw material over the surface of the nonwoven topsheet, adhering this absorbent material to the topsheet to that a glue has been pre-applied. Due to the molds with designs or patterns existing on the forming wheel and the vacuum action exerted from the vacuum system present in the core of this forming wheel, the distribution of the absorbent raw material will only occur in the designs or patterns in the molds, which will allow the adhesion of the Absorbent raw material to nonwoven topsheet with the shape of the designs or patterns existing in the molds and which happens by the suction exerted by the vacuum system.

This technique gives rise to a shield with an absorbent material distribution with the design pattern corresponding to the mold existing on the forming wheel, resulting in a underpad with a well-defined design or pattern transposed to it, as well as a product with greater softness compared to the absorbent underpads engraved using conventional embossing techniques.

In fact, the conventional technique, using embossing rolls for engraving designs or patterns on the underpad, gives rise to a product with the most compacted absorbent layer and with less definition of the designs or patterns, in addition to the space between designs being equally filled with absorbent material that is simply compressed by embossing and causes the existence of seams hardened by the crushing exerted by the compaction rollers or embossers in an undifferentiated way between the raw material and the backsheet.

Furthermore, this innovative technique also allows the same absorbent pad forming wheel to simultaneously produce pads designs or patterns, consecutively or with different alternately, according to the choice of these designs or patterns.

The conventional technique of engraving designs by embossing does not allow for this individualized and simultaneous customization of embossing, as the embossing roller can only execute on the guard the design or pattern that is engraved on it.

### BRIEF DESCRIPTION OF DRAWINGS

The present invention is described in detail below, without limitation and by way of example, by means of its preferred embodiment, represented in the accompanying drawings, in which:

FIG. 1 is a schematic and simplified representation of an embodiment of the process for executing the absorbent underpad on the forming wheel;

FIG. 2 is an exemplary graphic representation of a pattern or design to be transposed to the underpad;

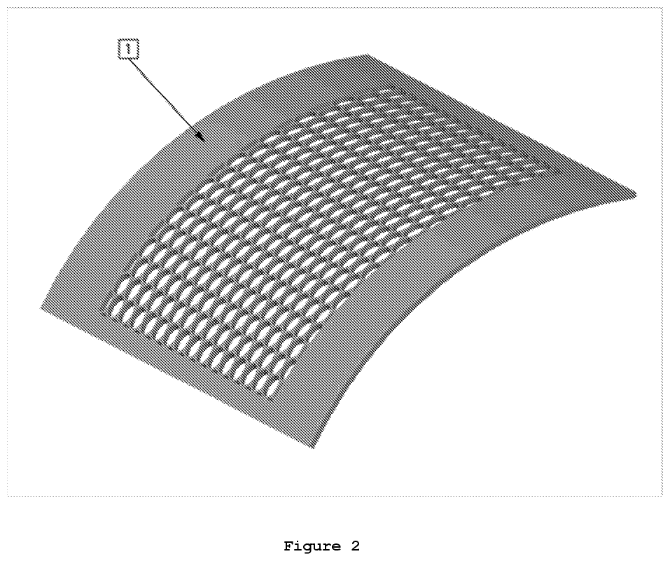

FIG. 3 is a simplified representation of the forming wheel with the molds of the drawings or patterns applied in the pocket of this forming wheel.

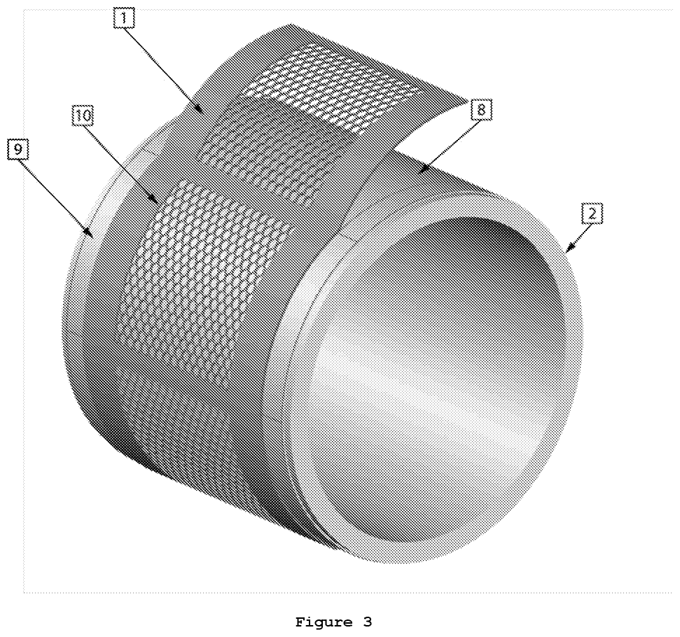

FIG. 4 is a graphical representation of the absorbent section of an absorbent underpad obtained by the process of transposing the design or pattern to the absorbent surface of the underpad.

FIG. 5 is a graphical representation of the backsheet of an absorbent underpad obtained by the process of transposing the design or pattern to the absorbent surface of the underpad.

Subtitle:

1—Mold with design or pattern;

2—Forming wheel;

3—Glue applicator;

4—Nonwoven topsheet;

5—Application of absorbent raw material;

6—Vacuum system;

7—Nonwoven topsheet with applied raw material;

8—Mesh or grid;

9—Pocket;

10—Mold with design or pattern (1) applied to the pocket (9) of the forming wheel (2);

11—Absorbent section of the underpad;

12—Drawing or pattern filled with absorbent raw material;

13—Perspective of the underpad backsheet;

14—Backsheet application (13) on the nonwoven topsheet with applied raw material (7).

### DETAILED DESCRIPTION OF THE INVENTION

The present invention reports to the production of an absorbent underpad with designs or patterns on its absorbent surface, using a technique that distributes the absorbent raw material through the pre-defined designs and patterns on a mold (1) applied to the forming wheel (2) of the underpads.

Referring to the figures, the preferred embodiment of the invention will now be described, which takes place in the underpad forming wheel (2), which consists of the set of organs and components represented in the referred figures, whose operation is described below.

The absorbent underpad with designs transposed by vacuum technique, whose patent is required, is produced in the forming wheel (2), which has pockets (9) in which the molds (1) with the desired and predefined designs or patterns are applied. The production process of the underpad with the transposition of the desired designs or patterns occurs in the forming Wheel (2), which is a component with a cylindrical shape and with a hollow interior, surrounded on the outside by a mesh or grid (8).

On the outer surface of this forming Wheel (2) in rotation, with the built-in molds (1) in the pockets (9) on the mesh or grid (8), the nonwoven topsheet (4) will pass with the glue previously and uniformly applied on its surface by the Glue Applicator (3).

When passing the nonwoven topsheet (4) on the forming Wheel (2), the absorbent raw material (5) will be deposited on the surface of the nonwoven topsheet (4) with the glue previously applied by the Glue Applicator (3).

At this point in the production process, for the absorbent raw material (5) to adhere to the surface of the nonwoven topsheet (4), this raw material will be subject to the vacuum action exerted by the vacuum system (6) installed on the forming wheel. (2).

As in the forming Wheel (2) in rotation and under the nonwoven topsheet (4) are built-in molds (1) with the pre-defined designs or patterns, which in turn are built-in in the respective pockets (10) on the mesh or grid (8), when the vacuum action is exerted by the vacuum system (6), suction of the absorbent raw material will occur through the mesh or grid (8) and the drawings or patterns cut in the molds (1).

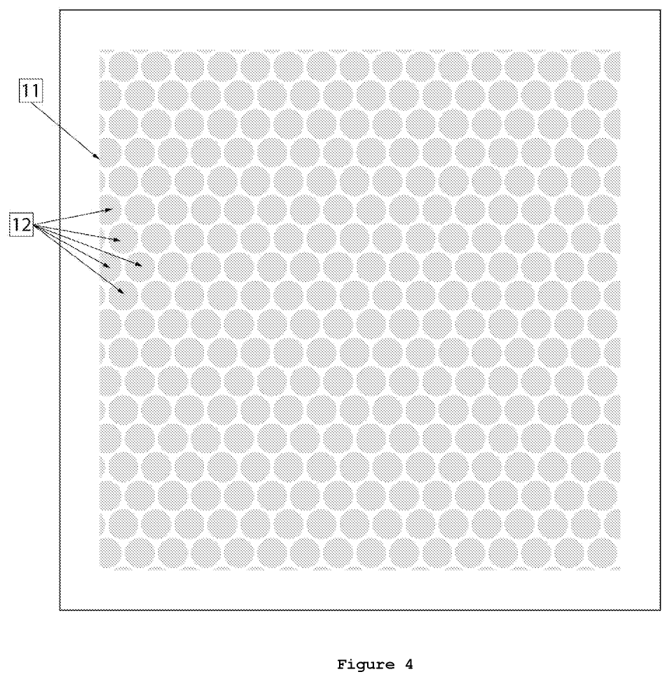

When this air suction occurs in the forming Wheel (2), this same air in production will pass through the mesh or grid (8), the designs or patterns cut in the cut-out molds (1) and the nonwoven topsheet (4), sucking the raw material applied (5) which will be glued to the nonwoven topsheet (4) and will be glued only to the gaps drawn in the molds (1) through which the air is sucked in, therefore assuming the shape of the design or pattern cut out in the molds (1), resulting in the absorbent section of the underpad (11) the design or pattern filled with absorbent raw material (12), resulting in an absorbent underpad obtained by this technique (FIG. 4) whose patent is required.

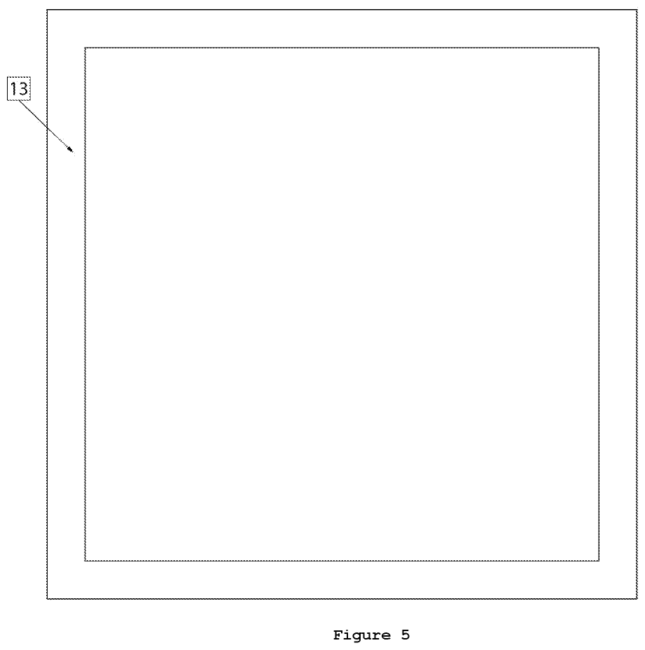

After the application of the absorbent raw material (5) on the nonwoven topsheet (4), which occurred on the forming wheel (2), this nonwoven topsheet with applied absorbent raw material (7) will proceed to the subsequent application of the backsheet (13) which corresponds to the impermeable part of the absorbent underpad (FIG. 5).

As the application of the backsheet (13) on the nonwoven topsheet with applied raw material (7) only occurs at a later stage (14) of the procedure for executing the designs or patterns on the absorbent underpad, there will be no transposition of the designs or patterns executed in the absorbent section (11) of the underpad (FIG. 4), to the backsheet (13) of the absorbent underpad, which corresponds to the impermeable part of it (FIG. 5).

The size of the underpad produced varies according to the number of molds (1) with the pre-defined designs or patterns to built-in in the pockets (9, 10) of the forming wheel (2).

In the same production process, molds (1) with different designs or patterns cut into them can be used simultaneously, resulting in absorbent underpads with the designs or patterns corresponding to those cut in the respective molds (1).

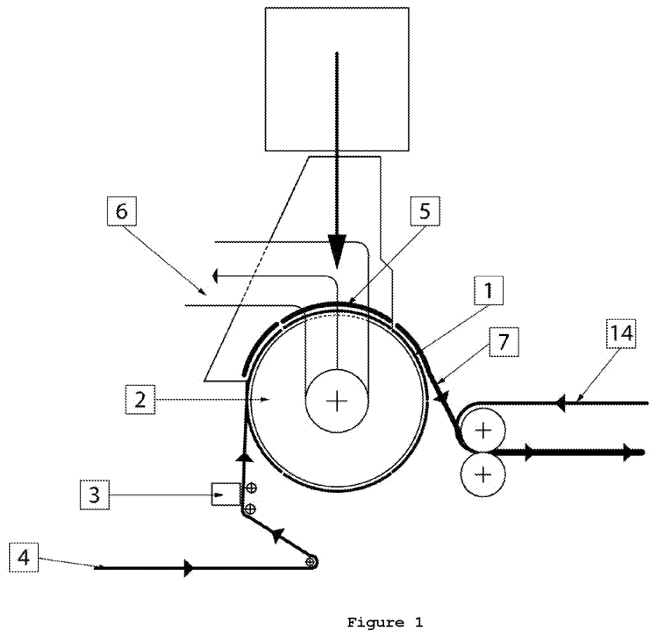

The forming wheel (2) is assembled on a support structure (not shown) that serves to house the other organs and components of the machine for the production of absorbent underpads (FIG. 1).

### INDUSTRIAL APPLICABILITY

The absorbent underpad with designs or patterns, obtained by the innovative method that is intended to be patented, in addition to presenting characteristics that differentiate it from the other existing absorbent underpads, namely regarding its smoothness in comparison to the underpads obtained by traditional embossing methods, also has a greater definition of the designs or patterns obtained by this technique.

1. Absorbent underpad (FIG. 4 and FIG. 5), with design or pattern transposed of cut-out molds (1) for the absorbent part of the underpad (11), characterized by defining the designs or patterns transposed;

2. Product according to claim 1, without engraving on the underpad backsheet (13) (FIG. 5);

3. Product according to claims 1 and 2, without compression or compaction of the absorbent section (11) of the absorbent underpad (FIG. 4) to obtain the drawings or patterns;

4. Product according to claims 1, 2 and 3, without engraving or crushing of nonwoven topsheet with applied raw material (7) and the backsheet (13) existing between the Drawings or patterns filled with absorbent raw material (12) obtained according to claims 1, 2 and 3 (FIG. 4);

5. Process of production of absorbent underpad with design or pattern transposed from cut-out molds (1) to absorbent section (11) of the absorbent underpad (FIGS. 4 and 5), understanding:
application of glue by the glue applicator (3) on the surface of the nonwoven topsheet (4);
placing of cut-out molds (1) with pre-defined designs or patterns, in the pockets (9) of the forming wheel (2) on the mesh or grid (8) (10);
passage of the nonwoven topsheet (4), with the glue pre-applied by the glue applicator (3), over cut-out molds (1) with the predefined drawings or patterns, in the pockets (10) of the forming wheel (2);
deposition of the absorbent raw material (5) on the surface of the nonwoven topsheet (4), with the glue pre-applied by the glue applicator (3);
subjection of the nonwoven topsheet (4) with the glue pre-applied by the glue applicator (3) and subsequent deposition of the absorbent raw material (5) the suction exerted by the vacuum system (6);
transposition of the designs or patterns in the cut molds (1) to the absorbent section (11) of the absorbent underpad (FIG. 4) by suction of the deposited raw material (5) through the cuted designs or patterns in the cut-out molds (1);
gluing on the nonwoven topsheet (4) of the absorbent raw material (5) occupying only the spaces cut-out in the molds (1) obtaining a design or pattern filled with absorbent raw material (12);
application of the backsheet (13) on the nonwoven topsheet with applied raw material (7), only after the stage following the procedure for executing the drawings or patterns on the absorbent underpad (14);

6. Production process of the absorbent underpad according to claim 5, characterized in that the dimension of the absorbent underpad (FIGS. 4 and 5) produced is conditioned to the size and quantity of cut-out molds (1) existing in the pockets (9, 10) of the Forming Wheel (2).

7. Production process of the absorbent underpad according to claims 5 and 6, characterized by the simultaneous existence (10) on the forming wheel (2) of molds (1) with different designs or patterns cut out on it, resulting in absorbent underpads with the drawings or patterns corresponding to those cut out in the respective molds (1).
